SEQUENCE LISTING (4 sequences)

SEQ ID NO: 1 - DNA, 27 bp, Artificial
  Synthetic oligonucleotide PCR primer.
cgagtacttcaatggcatcaagtacaa

SEQ ID NO: 2 - DNA, 27 bp, Artificial
  Synthetic oligonucleotide PCR primer.
ttcgtggtcgatgagttgcagcaggaa

SEQ ID NO: 3 - DNA, 26 bp, Artificial
  Synthetic oligonucleotide PCR primer.
tgaaggtcggtgtcaacggatttggc

SEQ ID NO: 4 - DNA, 24 bp, Artificial
  Synthetic oligonucleotide PCR primer.
catgtaggccatgaggtccaccac

(19) United States
(12) Patent Application Publication
(10) Pub. No.: US 2006/0024661 A1
(43) Pub. Date: Feb. 2, 2006

(54) Modulation of CRF potentiation of NMDA receptor currents via CRF receptor 2

(75) Inventors: Antonello Bonci, San Francisco, CA (US); Mark A. Ungless, London (GB)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 10/903,849

(22) Filed: Jul. 29, 2004

Related U.S. Application Data
(60) Provisional application No. 60/491,069, filed on Jul. 30, 2003.

(51) Int. Cl. C12Q 1/00; C12Q 1/68

(52) U.S. Cl. 435/4; 435/6

(57) ABSTRACT

This invention pertains to the discovery that CRF increases NMDAR (N-methyl-D-aspartate receptor)-mediated currents at excitatory synapses onto a subset of dopamine cells in the ventral tegmental area (VTA) in the mammalian brain. This effect is not blocked by a CRF receptor 1 (CRF-R1) antagonist, but is blocked by a CRF receptor 2 (CRF-R2) antagonist. It was also discovered that an inhibitor of the CRF-binding protein (CRF-BP) blocks the effects of CRF, which indicates that CRF-BP, rather than inactivating “free” CRF, is necessary for CRF to potentiate NMDAR currents. Methods are provided that exploit this discovery to screen for modulators (upregulators or downregulators) of NMDA potentiation by CRF.

### CROSS-REFERENCE TO RELATED APPLICATIONS

This application claims benefit of and priority to U.S. Ser. No. 60/491,069, filed on Jul. 30, 2003, which is incorporated herein by reference in its entirety for all purposes.

### STATEMENT AS TO RIGHTS TO INVENTIONS MADE UNDER FEDERALLY SPONSORED RESEARCH AND DEVELOPMENT

This work was supported in part by Grant Number 1R01DA15096-01 from the National Institute on Drug Abuse. The Government may have certain rights in this invention.

### FIELD OF THE INVENTION

This invention pertains to the field of neurobiology. In particular this invention pertains to the mechanism by which CRF potentiates activity at an NMDA receptor and to methods of screening for agents that modulate such potentiation.

### BACKGROUND OF THE INVENTION

Corticotrophin-releasing factor (CRF), a 41 amino acid peptide, plays an obligatory role in the activation of the hypothalamic-pituitary-adrenal axis and the subsequent release of glucocorticoids in response to stressful events (Koob and Heinrichs (1999) Brain Res. 848: 141-152; Kaufman et al. (2000) Biol. Psychiatry 48: 778-790; Behan et al. (1995) Nature 378: 284-287; Sarnyai et al. (2001) Pharmacol. Rev. 53: 209-243). In addition, extra-hypothalamic CRF mediates many behavioral responses to stress (Koob and Heinrichs (1999) Brain Res. 848: 141-152). Altered CRF levels are seen in a number of psychiatric and neurological disorders, such as depression and Alzheimer's disease (Kaufman et al. (2000) Biol. Psychiatry 48: 778-790; Behan et al. (1995) Nature 378: 284-287 (1995)). CRF is elevated in animal models of withdrawal from drugs of abuse and plays a key role in stress-induced relapse to drug taking (Sarnyai et al. (2001) Pharmacol. Rev. 53: 209-243). The cellular effects of CRF are mediated via two receptors (CRF-R1 and CRF-R2) (Dautzenberg and Hauger (2002) Trends Pharmacol. Sci. 23, 71-77); CRF also binds to a binding protein (CRF-BP), which is thought to inactivate ‘free’ CRF (Kemp et al. (1998) Peptides 19: 1119-1128). It has been suggested that CRF-BP inhibitors, which elevate ‘free’ CRF levels, may provide potential treatments for disorders where CRF levels are depressed, such as Alzheimer's disease and Parkinson's disease (Behan et al. (1995) Nature 378: 284-287). It is notable that many of these disorders involving elevated CRF levels are also thought to involve elevated dopamine levels and that CRF increases dopamine release in both limbic and cortical projection areas (Koob and Heinrichs (1999) Brain Res. 848: 141-152; Kaufman et al. (2000) Biol. Psychiatry 48: 778-790; Dunn and Berridge (1987) Pharmacol. Biochem. Behav. 27: 685-691). How CRF modulates dopaminergic activity, however, is unclear.

Dopamine neurons in the ventral tegmental area (VTA) are under important regulatory control from excitatory glutamatergic projections from a number of brain regions, such as the prefrontal cortex and amygdala, and modulation of these synapses is involved in both short- and long-term changes in dopaminergic activity (Bonci and Malenka (1999) J Neurosci. 19: 3723-3730; Overton et al. (1999) Neuroreport. 10: 221-226; Ungless et al. (2001) Nature 411: 584-587). In particular, N-methyl-D-aspartate receptors (NMDARs) play a key role in regulating burst firing and the induction of long-term synaptic potentiation in these neurons (8. Bonci and Malenka (1999) J Neurosci. 19: 3723-3730; Overton et al. (1999) Neuroreport. 10: 221-226; Ungless et al. (2001) Nature 411: 584-587; Overton and Clark (1997) Brain Res. Brain Res. Rev. 25: 312-334). Interestingly, stress-induced activation of the dopamine system requires NMDAR activity (Morrow et al. (1993) Eur. J. Pharmacol. 238: 255-262), and repeated stress induces increases in NMDAR and α-amino-3-hydroxy-5-methyl-isoxazolepropionic acid receptor (AMPAR) subunits in the VTA (Fitzgerald et al. (1996) J Neurosci. 16: 274-282). Although CRF has been shown to modulate neuronal excitability in a number of brain regions (Aldenhoff et al. (1983) Science 221: 875-877; Valentino et al. (1983) Brain Res. 270: 363-367), increase field potentials and prime population-spike long-term potentiation (LTP) in the hippocampus (Wang et al. (1998) Eur. J. Neurosci. 10: 3428-3437; Blank et al. (2002) J. Neurosci. 22: 3788-3794), its role in the modulation of excitatory synaptic transmission is poorly understood.

### SUMMARY OF THE INVENTION

This invention pertains to the discovery that CRF increases NMDAR (N-methyl-D-aspartate receptor)-mediated currents at excitatory synapses onto a subset of dopamine cells in the ventral tegmental area (VTA) in the mammalian brain. This effect is not blocked by a CRF receptor 1 (CRF-R1) antagonist, but is blocked by a CRF receptor 2 (CRF-R2) antagonist. It was also discovered that an inhibitor of the CRF-binding protein (CRF-BP) blocks the effects of CRF, which indicates that CRF-BP, rather than inactivating “free” CRF, is necessary for CRF to potentiate NMDAR currents.

Methods are provided that exploit this discovery to screen for modulators (upregulators or downregulators) of NMDA potentiation by CRF.

Definitions

The term “substance of abuse” refers to a substance that is psychoactive and that induces tolerance and/or addiction. Substances of abuse include, but are not limited to stimulants (e.g. cocaine, amphetamines), opiates (e.g. morphine, heroin), cannabinoids (e.g. marijuana, hashish), nicotine, alcohol, substances that mediate agonist activity at the dopamine D2 receptor, and the like. Substances of abuse include, but are not limited to addictive drugs.

The term “gene product” refers to a molecule that is ultimately derived from a gene. The molecule can be a polypeptide encoded by the gene, an mRNA encoded by a gene, a cDNA reverse transcribed from the mRNA, and so forth.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The term “antibody”, as used herein, includes various forms of modified or altered antibodies, such as an intact immunoglobulin, an Fv fragment containing only the light and heavy chain variable regions, an Fv fragment linked by a disulfide bond (Brinkmann et al. (1993) Proc. Natl. Acad. Sci. USA, 90: 547-551), an Fab or (Fab)′2 fragment containing the variable regions and parts of the constant regions, a single-chain antibody and the like (Bird et al. (1988) Science 242: 424-426; Huston et al. (1988) Proc. Nat. Acad. Sci. USA 85: 5879-5883). The antibody may be of animal (especially mouse or rat) or human origin or may be chimeric (Morrison et al. (1984) Proc Nat. Acad. Sci. USA 81: 6851-6855) or humanized (Jones et al. (1986) Nature 321: 522-525, and published UK patent application #8707252).

The terms “binding partner”, or “capture agent”, or a member of a “binding pair” refers to molecules that specifically bind other molecules to form a binding complex such as antibody-antigen, lectin-carbohydrate, nucleic acid-nucleic acid, biotin-avidin, etc.

The term “specifically binds”, as used herein, when referring to a biomolecule (e.g., protein, nucleic acid, antibody, etc.), refers to a binding reaction which is determinative of the presence biomolecule in heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 141 9), phosphorothioate (Mag et al. (1991) Nucleic Acids Res. 19:1437; and U.S. Pat. No. 5,644,048), phosphorodithioate (Briu et al. (1989) J. Am. Chem. Soc. 111 :2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside &Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic &Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences. Stringent hybridization and stringent hybridization wash conditions in the context of nucleic acid hybridization are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, chapt 2, Overview of principles of hybridization and the strategy of nucleic acid probe assays, Elsevier, N.Y. (Tijssen). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook (1989) Molecular Cloning: A Laboratory Manual (2nd ed.) Vol. 1-3, Cold Spring Harbor Laboratory, Cold Spring Harbor Press, NY, and detailed discussion, below), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook supra.) for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 40° C. for 15 minutes.

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term database refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

The phrase “expression or activity of a gene” (e.g. Tsp42Ee gene) refers to the production of a gene product (e.g. the production of an mRNA and/or a protein) or to the activity of a gene product (i.e., the activity of a protein encoded by the gene).

The term “expression” refers to protein expression, e.g., mRNA and/or translation into protein. The term “activity” refers to the activity of a protein. Activities include but are not limited to phosphorylation, signaling activity, activation, catalytic activity, protein-protein interaction, transportation, etc. The expression and/or activity can increase, or decrease. Expression and/or activity can be activated or inhibited directly or indirectly.

A “CRF, and/or CRF-BP, and/or CRF2 nucleic acid or polypeptide” refers to a polypeptide that is CRF, CRF-BP or cRF2 and/or to fragments thereof and/or two nucleic acids that encode the CRF, and/or CRF-BP, and/or CRF2 and/or to nucleic acids derived therefrom.

The term “detecting”, particularly when used with reference to electrophysiological methods includes, but is not limited to recording an electrophysiological signal from one or more cells.

### BRIEF DESCRIPTION OF THE DRAWINGS

FIGS. 1A, 1B, 1C, and 1D show that CRF potentiates NMDAR-mediated synaptic transmission in dopamine neurons with a large Iₕ. Filled circles correspond to large Iₕ neurons and open circles correspond to small Iₕ neurons. FIG. 1A: Examples of the effect of CRF on NMDAR-mediated EPSCs in two dopamine neurons. Scale bars: 20 pA, 40 msec. FIG. 1: Examples of Iₕ from both types of neuron. Iₕ is computed as the difference between time points 1 and 2. FIG. 1C: Large Iₕ neurons exhibit CRF-induced potentiation of NMDAR-mediated EPSCs. Small Iₕ neurons do not exhibit CRF-induced potentiation. Left axis represents the magnitude of the effect of CRF on NMDAR-mediated EPSCs (20-30 min), plotted against Iₕ size on the horizontal. Right axis represents frequency of cells in 10 pA bins of Iₕ. Dopamine neurons form a bimodal distribution of Iₕ size, which corresponds to the CRF and non-CRF responsive groups. FIG. 1D, upper panel: Summary of responses to CRF in large Iₕ (n=7) and small Iₕ (n=8) neurons. FIG. 1D, lower panel: CRF does not potentiate AMPAR-mediated EPSCs. Scale bars: 40 pA, 5 msec (n=4). *=P<0.05 vs. baseline.

FIGS. 2A, 2B, 2C, 2D, and 2E show that CRF-induced potentiation of NMDAR-mediated synaptic transmission requires CRF receptor 2 and phospholipase C. Filled bars denote CRF application; Open bars denote application of antagonist in title of each panel. FIG. 2A: CRF-R1 antagonist, CP-154,526, does not block CRF-induced potentiation of NMDAR-mediated EPSCs (n=6). FIG. 2B: CRF-R2 antagonist, Antisauvagine-30, blocks CRF-induced potentiation of NMDAR-mediated EPSCs (n=5). FIG. 2C: Total RNAs from VTA, septum and cerebellum were isolated and the expression of CRF-R2 and control GPDH were analyzed by RT-PCR as described in methods. PCR products were separated on an agarose gel and photographed by Eagle Eye II. Results are representative of three independent experiments. FIG. 2D: CRF-induced potentiation of NMDAR-mediated EPSCs is not blocked by the cAMP inhibitor, Rp-cAMPs (n=4). FIG. 2E: CRF-induced potentiation of NMDAR-mediated EPSCs is blocked by the phospholipase C inhibitor, U73122 (n=5). *=P<0.05 vs. baseline.

FIGS. 3A, 3B, 3C, 3D, 3E, and 3F show that CRF-binding protein is necessary for CRF-induced potentiation of NMDAR-mediated synaptic transmission. Filled bars denote CRF application; Open bars denote application of substance in title of each panel. FIG. 3A: CRF fragment (6-33), which competes with CRF at the CRF-BP, but does not bind CRF-R2, inhibits CRF-induced potentiation of NMDAR-mediated EPSCs (n=5). FIG. 3B: bovine CRF, which binds with high affinity to CRF-R2, but not CRF-BP, does not induce potentiation of NMDAR-mediated EPSCs (n=5). FIG. 3C: Urocortin potentiates NMDARs in cells with large Iₕ (filled circles) (n=6), but not those with small Iₕ (open circles) (n=3). FIG. 3D: Urocortin II does not potentiate NMDARs (n=4). FIGS. 3E, F: A diagram showing the interaction between the CRF system and NMDARs in dopamine neurons. (FIG. 3E), when CRF is not bound to CRF-BP, it does not activate CRF-R2. (FIG. 3F). When CRF is bound to CRF-BP it can activate CRF-R2 and the PLC pathway to potentiate NMDARs in dopamine neurons. *=P<0.05 vs. baseline.

### DETAILED DESCRIPTION

Corticotrophin-releasing factor (CRF) plays a central role in physiological responses to stress. It is also implicated in a broad range of disorders, such as depression, Alzheimer's disease and drug abuse. Despite its important role in behavioral responses to stress and in various disease states, the role of CRF in the modulation of synaptic activity has been poorly understood.

This invention pertains to the discovery that CRF increases NMDAR (N-methyl-D-aspartate receptor)-mediated currents at excitatory synapses onto a subset of dopamine cells in the ventral tegmental area (VTA) in the mammalian brain. This effect is not blocked by a CRF receptor 1 (CRF-R1) antagonist, but is blocked by a CRF receptor 2 (CRF-R2) antagonist. It was also discovered that an inhibitor of the CRF-binding protein (CRF-BP) blocks the effects of CRF, which indicates that CRF-BP, rather than inactivating “free” CRF, is necessary for CRF to potentiate NMDAR currents. Accordingly, Urocortin, which may be the endogenous CRF-R2 ligand and also binds CRF-BP, mimics CRF, while ovine CRF and Urocortin II, which do not bind CRF-BP, do not potentiate NMDAR currents. These results provide the first specific roles for CRF-R2 and CRF-BP in the modulation of neuronal activity.

These discoveries can be exploited in a wide variety of contexts. For example, in view of these discoveries, corticotrophin-releasing factor (CRF) provides a good target for agents that modulate the potentiation of N-methyl-D-aspartate receptor (NMDAR) mediated currents. Such agents can be used, for example, in the treatment of substance abuse (e.g., self-administration of substances of abuse) and/or withdrawal from substances of abuse, and various neurological conditions characterized by overactivation, inactivation, and/or loss of dopinergic neurons (e.g. Alzheimer's disease, Parkinson's disease, etc.). Thus, in certain embodiments, this invention provides methods of screening for agent(s) that modulate corticotrophin-releasing factor (CRF) potentiation of N-methyl-D-aspartate receptor (NMDAR) mediated currents. The methods typically involve contacting a cell, tissue or organism with one or more test agents and detecting the activity or expression of a CRF2 receptor, where an alteration of expression or activity of a CRF2 receptor as compared to a control indicates that said test agent is an agent that modulates CRF potentiation of NMDAR-mediated currents and is a good candidate compound for use in the treatment of substance abuse, withdrawal, and a variety of other conditions, e.g. as described herein.

In addition, it was a surprising discovery that CRF binding protein (CRF-BP), rather than inactivating ‘free’ CRF, is necessary for CRF to potentiate NMDAR currents and this potentiation is mediated via the CRF2 receptor, not the CRF1 receptor. Thus CRF, and CRF-BP both appear to be required to activate/potentiate NMDA receptors. Thus, the interaction between these three components (CRF, CRF-BP, and the CRF2 receptor) also provide effective targets for screening for modulators of NMDA potentiation.

Without being bound to a particular theory, it is believed that this CRF/CRF-BP/CRF2R interaction provides a previously unknown link between CRF and dopamine activity. It is believed that CRF2R agonists can provide a therapeutic modality for Parkinson's and/or Alzheimers disease (or other related pathologies) by activating NMDA receptors. In certain embodiments, such agonists will directly agonize/activate CRF2 receptors. In certain other embodiments, such agonists will act by binding both CRF2 receptors and CRF-BP. To our knowledge, this is first experimental evidence for a role of CRF 2 receptors on any form of synaptic transmission in the brain. Furthermore, we believe this is the first demonstration that CRF binding protein is a component in determining the functional outcome of CRF receptor 2 activation.

Without being bound to a particular theory, we believe that an agonist at CRF2 receptors that, in certain embodiments, also bind to the CRF-binding protein will act as a cognitive enhancer. CRF plays a clear role in physiological brain processes such as learning, but also in disorders such as depression, Alzheimer's disease, Parkinson's disease, and drug abuse. We believe that such a compound will provide a novel therapeutic agent in these disorders, where a cognitive impairment has been observed.

Conversely, in conditions characterized by elevated CRF (e.g., addiction, chronic stress, etc.) agents can be used that inhibit CRF2 activation by acting at the CRF2 receptor and/or at the CRF/CRF-BP/CRF2 receptor interaction.

Such CRF2R agonists or antagonists can be identified by screening for activity Such agents can be identified by screening for the ability to upregulate or inhibit expression or activity of the CRF2 receptor and/or CRF and/or CRF-BP and or the interaction of these components (e.g. by binding CRF, CRF-BP, CRF2R or a complex of two or more of these proteins).

Thus, in one embodiment, this invention provides a method of screening for an agent that modulates the activity of corticotrophin-releasing factor (CRF) on a dopaminergic neuron. The methods involve contacting a test agent to one or more components of CRF signaling (e.g. CRF, a CRF-BP, CRF2 receptor) and detecting an increase or decrease in interaction between CRF and CRF BP and/or CRF and the receptor where an increase or decrease in the interaction, e.g., as compared to a control, indicates that the test agent modulates the activity of corticotrophin-releasing factor (CRF) on a dopaminergic neuron.

Using known agents, and/or agents identified in the screening methods described herein a variety of therapeutic modalities can be achieved. Thus, for example, this invention provides a method of modulating the activity of corticotrophin-releasing factor (CRF) on a dopaminergic neuron. The method involves modulating (e.g., increasing or decreasing) binding between CRF and CRF-BP and/or modulating binding between CRF and the CRF2 receptor. Similarly, also provides is a method of mitigating one or more symptoms associated with chronic consumption of a substance of abuse or with withdrawal from such chronic consumption. The method typically involves inhibiting interaction between CRF and CRFBP1 and/or a CRF2 receptor. Conversely, long term potentiation (e.g., mitigating a symptom associated with Alzheimer's disease, Parkinson's disease and the like) can be enhanced by enhancing interaction between CRF and CRFBP1 and/or a CRF2 receptor.

I. Assays for Agents that Modulate CRF Potentiation of NMDA Receptors.

As indicated above, in one aspect, this invention pertains to the discovery of a mechanism underlying CRF potentiation of NMDA receptors. The effect is mediated via the interaction of CRF, CRF binding protein (CRF-BP), and the CRF2 receptor. Thus, agents that modulate the interaction of CRF and/or CRF-BP and/or CRF2R and/or that modulate (e.g., upregulate and/or downregulate) the expression and/or activity of CRF and/or CRF-BP and/or CRF2R expected to have prophylactic and/or therapeutic utility as described herein. Thus, In certain embodiments this invention provides methods of screening for agents that modulate the interaction, activity, and/or expression or expression of CRF and/or CRF-BP and/or CRF2 receptor.

The methods typically involve direct assays for the interaction of CRF and/or CRF-BP and/or the CRF2 receptor or detecting the activity of CRF2 receptor or potentiation of NMDA receptors and/or detecting alterations in the expression level and/or activity level of a CRF, CRF-BP, and/or CRF2 receptor genes or gene products caused by the treatment with one or more of the agent(s) in question. An elevated expression level or activity level produced by the agent as, e.g., compared to a negative control where the test agent is absent or at reduced concentration indicates that the agent upregulates activity or expression of the factor(s) in question. Conversely, decreased expression level or activity level resulting from treatment by the agent as compared to a negative control where the test agent is absent or at reduced concentration indicates that the agent down-regulates expression or activity of the factor(s).

A) Assaying for Modulators of Protein Interaction.

In certain embodiments, this invention pertains to assays for agents that modulate the interaction of CRF and/or CRF-BP and/or CRF2 receptor and thereby agonize or antagonize CRF activity at the CRF2 receptor. In certain embodiments this involves contacting a cell, tissue, or organism with one or more test agents and evaluating the effect of the test agent(s) on the interaction of CRF, CRF-BP, and/or CRF2 receptor. Methods of screening for the effect of test agents on protein/protein interactions are well known to those of skill in the art. Such methods include, but are not limited to two-hybrid systems, gel-shift assays, and the like.

In a two hybrid system two chimeric molecules are created one of which bears a nucleic acid binding region, the other of which bears an expression control element (e.g. a transactivation or repressor domain). The molecules each further comprise one of the two proteins whose interaction is to be assayed. The chimeric molecule comprising the DNA binding domain binds to a “substrate nucleic acid. When the two proteins of interest interact/bind, i.e., domain of the chimeric molecule recognizes and binds to its cognate binding partner on the second chimeric molecule thereby recruiting that molecule to the nucleic acid whereby the expression control element alters (e.g. activates) expression of a gene or cDNA comprising the underlying nucleic acid. This provides a detectable signal that is an indicator of ht protein/protein interaction. The effect of one or more test agent(s) on this interaction can then readily be evaluated. Two-hybrid systems are well known to those of skill in the art (see, e.g., Fields and Song (1989) Nature 340: 245-246).

In a gel-shift assay one or more of the proteins whose biding is to be evaluated is labeled with a detectable label. Where the proteins bind to each other the mobility of the complex thus formed is different than the mobility of the individual component proteins and can readily be detected (e.g. in an electrophoretic gel). The effect of one or more test agents on the formation of such complexes can then readily be detected.

These assays are intended to be illustrative and not limiting. Using the teaching provided herein, numerous other assays for evaluating the effect of one or more test agents on CRF, CRF-BP and CRF2R interaction can readily be provided.

B) Assaying for Modulators of Activity.

In one embodiment, the effect of one or more test agents on CRF-BP, CRF and/or CRF2 receptor activity can be directly evaluated. In one such approach the test agent(s) are contacted to a neurological tissue preparation (e.g. a brain slice preparation) and the effect of the test agent on CRF potentiation of NMDA receptor currents is evaluated using electrophysiological techniques as described herein in Example 1.

C) Assaying for Modulators of CRF, CRF-BP, and/or CRF2R Expression.

Expression levels of a gene can be altered by changes in by changes in the transcription of the gene product (i.e. transcription of mRNA), and/or by changes in translation of the gene product (i.e. translation of the protein), and/or by post-translational modification(s) (e.g. protein folding, glycosylation, etc.). Thus preferred assays of this invention typically contacting a test cell, tissue, or animal with one or more test agents, and assaying for level of transcribed mRNA (or other nucleic acids derived from the neurotrophic and/or neurogenerative factor gene(s)), level of translated protein, activity of translated protein, etc. Examples of such approaches are described below.

1) Nucleic-Acid Based Assays.

a. Target Molecules.

Changes in expression level can be detected by measuring changes in mRNA and/or a nucleic acid derived from the mRNA (e.g. reverse-transcribed cDNA, etc.). In order to measure the CRF, and/or CRF-BP, and/or CRF2 receptor expression level it is desirable to provide a nucleic acid sample for such analysis. In preferred embodiments the nucleic acid is found in or derived from a biological sample. The term “biological sample”, as used herein, refers to a sample obtained from an organism or from components (e.g., cells) of an organism. The sample may be of any biological tissue or fluid. Biological samples may also include organs or sections of tissues such as frozen sections taken for histological purposes.

The nucleic acid (e.g., mRNA nucleic acid derived from mRNA) is, in certain preferred embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In a preferred embodiment, the “total” nucleic acid is isolated from a given sample using, for example, an acid guanidinium-phenol-chloroform extraction method and polyA+ mRNA is isolated by oligo dT column chromatography or by using (dT)ₙ magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to assaying for expression level. Methods of amplifying nucleic acids are well known to those of skill in the art and include, but are not limited to polymerase chain reaction (PCR, see. e.g., Innis, et al., (1990) PCR Protocols. A guide to Methods and Application. Academic Press, Inc. San Diego,), ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.).

In a particularly preferred embodiment, where it is desired to quantify the transcription level (and thereby expression) of factor(s) of interest in a sample, the nucleic acid sample is one in which the concentration of the Tsp42Ee, and/or a Tsp42E1, and/or an sgg transcript, or the concentration of the nucleic acids derived from the Tsp42Ee, and/or a Tsp42E1, and/or an sgg mRNA transcript(s), is proportional to the transcription level (and therefore expression level) of that gene. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes.

Where more precise quantification is required, appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript or large differences of changes in nucleic acid concentration is desired, no elaborate control or calibration is required.

In the simplest embodiment, the nucleic acid sample is the total mRNA or a total cDNA isolated and/or otherwise derived from a biological sample (e.g. a sample from a neural cell or tissue). The nucleic acid may be isolated from the sample according to any of a number of methods well known to those of skill in the art as indicated above.

b. Hybridization-Based Assays.

Using the known sequence of CRF, and/or CRF-BP, and/or CRF2 receptor detecting and/or quantifying the transcript(s) can be routinely accomplished using nucleic acid hybridization techniques (see, e.g., Sambrook et al. supra). For example, one method for evaluating the presence, absence, or quantity of reverse-transcribed cDNA involves a “Southern Blot”. In a Southern Blot, the DNA (e.g., reverse-transcribed CRF, and/or CRF-BP, and/or CRF2 receptor mRNA), typically fragmented and separated on an electrophoretic gel, is hybridized to a probe specific for the nucleic acid encoding the CRF, and/or CRF-BP, and/or CRF2 receptor. Comparison of the intensity of the hybridization signal from the target specific probe with a “control” probe (e.g. a probe for a “housekeeping gene) provides an estimate of the relative expression level of the target nucleic acid.

Alternatively, the CRF, and/or CRF-BP, and/or CRF2 receptor mRNA can be directly quantified in a Northern blot. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify and/or quantify the target mRNA. Appropriate controls (e.g. probes to housekeeping genes) provide a reference for evaluating relative expression level.

An alternative means for determining the CRF, and/or CRF-BP, and/or CRF2 receptor expression level is in situ hybridization. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

In some applications it is necessary to block the hybridization capacity of repetitive sequences. Thus, in some embodiments, tRNA, human genomic DNA, or Cot-1 DNA is used to block non-specific hybridization.

c. Amplification-Based Assays.

In another embodiment, amplification-based assays can be used to measure CRF, and/or CRF-BP, and/or CRF2 receptor expression (transcription) level. In such amplification-based assays, the target nucleic acid sequences (i.e., CRF, and/or CRF-BP, and/or CRF2 receptor nucleic acid(s)) act as template(s) in amplification reaction(s) (e.g. Polymerase Chain Reaction (PCR) or reverse-transcription PCR (RT-PCR)). In a quantitative amplification, the amount of amplification product will be proportional to the amount of template in the original sample. Comparison to appropriate (e.g. healthy tissue or cells unexposed to the test agent) controls provides a measure of the CRF, and/or CRF-BP, and/or CRF2 receptor transcript level.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). One approach, for example, involves simultaneously co-amplifying a known quantity of a control sequence using the same primers as those used to amplify the target. This provides an internal standard that may be used to calibrate the PCR reaction.

One preferred internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of labeled nucleic acid (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al. (1990) Academic Press, Inc. N.Y. The known nucleic acid sequence(s) for CRF, and/or CRF-BP, and/or CRF2 receptor, and/or sgg are sufficient to enable one of skill to routinely select primers to amplify any portion of the gene.

d. Hybridization Formats and Optimization of Hybridization

i. Array-Based Hybridization Formats.

In one embodiment, the methods of this invention can be utilized in array-based hybridization formats. Arrays are a multiplicity of different “probe” or “target” nucleic acids (or other compounds) attached to one or more surfaces (e.g., solid, membrane, or gel). In a preferred embodiment, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

This simple spotting, approach has been automated to produce high density spotted arrays (see, e.g., U.S. Pat. No. 5,807,522). This patent describes the use of an automated system that taps a microcapillary against a surface to deposit a small volume of a biological sample. The process is repeated to generate high density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high density arrays is also described in U.S. Pat. Nos. 5,744,305, 5,800,992 and 5,445,934.

ii. Other Hybridization Formats

As indicated above a variety of nucleic acid hybridization formats are known to those skilled in the art. For example, common formats include sandwich assays and competition or displacement assays. Such assay formats are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ^{±}S, ¹⁴C, or ³²P-labelled probes or the like. Other labels include ligands that bind to labeled antibodies, fluorophores, chemi-luminescent agents, enzymes, and antibodies which can serve as specific binding pair members for a labeled ligand.

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

e. Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25× SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

f. Labeling and Detection of Nucleic Acids.

The probes used herein for detection of CRF, and/or CRF-BP, and/or CRF2 receptor expression levels can be full length or less than the full length of the CRF, and/or CRF-BP, and/or CRF2 receptor mRNA(s). Shorter probes are empirically tested for specificity. Preferred probes are sufficiently long so as to specifically hybridize with the target nucleic acid(s) under stringent conditions. The preferred size range is from about 20 bases to the length of CRF, and/or CRF-BP, and/or CRF2 receptor mRNA, more preferably from about 30 bases to the length of the CRF, and/or CRF-BP, and/or CRF2 receptor mRNA, and most preferably from about 40 bases to the length of CRF, and/or CRF-BP, and/or CRF2 receptor mRNA.

The probes are typically labeled, with a detectable label. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another.

Suitable chromogens which can be employed include those molecules and compounds which absorb light in a distinctive range of wavelengths so that a color can be observed or, alternatively, which emit light when irradiated with radiation of a particular wave length or wave length range, e.g., fluorescers.

Desirably, fluorescent labels should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye can differ from the unbound dye. Therefore, when referring to the various wavelength ranges and characteristics of the dyes, it is intended to indicate the dyes as employed and not the dye which is unconjugated and characterized in an arbitrary solvent.

Detectable signal can also be provided by chemiluminescent and bioluminescent sources. Chemiluminescent sources include a compound which becomes electronically excited by a chemical reaction and can then emit light which serves as the detectable signal or donates energy to a fluorescent acceptor. Alternatively, luciferins can be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

The label can be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are easily added during an in vitro transcription reaction. Thus, for example, fluorescein labeled UTP and CTP can be incorporated into the RNA produced in an in vitro transcription.

The labels can be attached directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe-CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

2) Polypeptide-Based Assays.

The CRF, and/or CRF-BP, and/or CRF2 receptor polypeptide(s) can be detected and quantified by any of a number of methods well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In one preferred embodiment, the CRF, and/or CRF-BP, and/or CRF2 receptor polypeptide(s) are detected/quantified in an electrophoretic protein separation (e.g. a 1- or 2-dimensional electrophoresis). Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another preferred embodiment, Western blot (immunoblot) analysis is used to detect and quantify the presence of polypeptide(s) of this invention in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target polypeptide(s) and may be directly labeled or alternatively may be subsequently detected using labeled antibodies (e.g., labeled sheep anti-mouse antibodies) that specifically bind to the a domain of the antibody.

In preferred embodiments, the CRF, and/or CRF-BP, and/or CRF2 receptor polypeptide(s) are detected using an immunoassay. As used herein, an immunoassay is an assay that utilizes an antibody to specifically bind to the analyte (e.g., the target polypeptide(s)). The immunoassay is thus characterized by detection of specific binding of a polypeptide of this invention to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (e.g., CRF, and/or CRF-BP, and/or CRF2 receptor proteins). In preferred embodiments, the capture agent is an antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/polypeptide complex.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Preferred immunoassays for detecting the target polypeptide(s) are either competitive or noncompetitive. Noncompetitive immunoassays are assays in which the amount of captured analyte is directly measured. In one preferred “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte (e.g. CRF, and/or CRF-BP, and/or CRF2 receptor protein) present in the sample is measured indirectly by measuring the amount of an added (exogenous) analyte displaced (or competed away) from a capture agent (antibody) by the analyte present in the sample. In one competitive assay, a known amount of, in this case, labeled polypeptide is added to the sample and the sample is then contacted with a capture agent. The amount of labeled polypeptide bound to the antibody is inversely proportional to the concentration of target polypeptide present in the sample.

In one particularly preferred embodiment, the antibody is immobilized on a solid substrate. The amount of target polypeptide bound to the antibody may be determined either by measuring the amount of target polypeptide present in an polypeptide/antibody complex, or alternatively by measuring the amount of remaining uncomplexed polypeptide.

The immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies that bind CRF, and/or CRF-BP, and/or CRF2 receptor, either alone or in combination. In the case where the antibody that binds the CRF, and/or CRF-BP, and/or CRF2 receptor polypeptide(s) is not labeled, a different detectable marker, for example, an enzyme-labeled antibody capable of binding to the monoclonal antibody which binds the CRF, and/or CRF-BP, and/or CRF2 receptor polypeptide, can be employed. Any of the known modifications of EIA, for example, enzyme-linked immunoabsorbent assay (ELISA), may also be employed. As indicated above, also contemplated by the present invention are immunoblotting immunoassay techniques such as western blotting employing an enzymatic detection system.

The immunoassay methods of the present invention can also include other known immunoassay methods, for example, fluorescent immunoassays using antibody conjugates or antigen conjugates of fluorescent substances such as fluorescein or rhodamine, latex agglutination with antibody-coated or antigen-coated latex particles, haemagglutination with antibody-coated or antigen-coated red blood corpuscles, and immunoassays employing an avidin-biotin or strepavidin-biotin detection systems, and the like.

The particular parameters employed in the immunoassays of the present invention can vary widely depending on various factors such as the concentration of antigen in the sample, the nature of the sample, the type of immunoassay employed and the like. Optimal conditions can be readily established by those of ordinary skill in the art. In certain embodiments, the amount of antibody that binds the CRF, and/or CRF-BP, and/or CRF2 receptor polypeptide is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2M sodium chloride, preferably about that of 0.15M sodium chloride. Times will vary widely depending upon the nature of the assay, and generally range from about 0.1 minute to about 24 hours. A wide variety of buffers, for example PBS, may be employed, and other reagents such as salt to enhance ionic strength, proteins such as serum albumins, stabilizers, biocides and non-ionic detergents can also be included.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein, are commercially available or can be produced using standard methods well know to those of skill in the art.

It will also be recognized that antibodies can be prepared by any of a number of commercial services (e.g., Berkeley antibody laboratories, Bethyl Laboratories, Anawa, Eurogenetec, etc.).

3) Assay Optimization.

The assays of this invention have immediate utility in screening for agents that modulate the expression or activity of CRF, and/or CRF-BP, and/or CRF2 receptor by a cell, tissue or organism. The assays of this invention can be optimized for use in particular contexts, depending, for example, on the source and/or nature of the biological sample and/or the particular test agents, and/or the analytic facilities available. Thus, for example, optimization can involve determining optimal conditions for binding assays, optimum sample processing conditions (e.g. preferred PCR conditions), hybridization conditions that maximize signal to noise, protocols that improve throughput, etc. In addition, assay formats can be selected and/or optimized according to the availability of equipment and/or reagents. Thus, for example, where commercial antibodies or ELISA kits are available it may be desired to assay protein concentration. Conversely, where it is desired to screen for modulators that alter transcription the CRF, and/or CRF-BP, and/or CRF2 receptor gene(s), nucleic acid based assays are preferred.

Routine selection and optimization of assay formats is well known to those of ordinary skill in the art.

II. Pre-Screening for Agents that Bind a CRF and/or CRF-BP, and/or CRF2R and/or Complexes Thereof.

In certain embodiments it is desired to pre-screen test agents for the ability to interact with (e.g. specifically bind to) CRF and/or CRF-BP, and/or CRF2R and/or complexes thereof or to a nucleic acid encoding CRF, CRF-BP, and/or CRF2R. Specifically binding test agents are more likely to interact with one or more of these components and thereby modulate CRF potentiation of NMDA receptors. Thus, in some preferred embodiments, the test agent(s) are pre-screened for binding to CRF and/or CRF-BP, and/or CRF2R and/or complexes thereof or to a nucleic acid encoding CRF, CRF-BP, and/or CRF2R before performing the more complex assays described above.

In one embodiment, such pre-screening is accomplished with simple binding assays. Means of assaying for specific binding or the binding affinity of a particular ligand for a nucleic acid or for a protein are well known to those of skill in the art. In preferred binding assays, the CRF and/or CRF-BP, and/or CRF2R and/or complexes thereof or the nucleic acid encoding CRF, CRF-BP, and/or CRF2R is immobilized and exposed to a test agent (which can be labeled), or alternatively, the test agent(s) are immobilized and exposed to CRF and/or CRF-BP, and/or CRF2R and/or complexes thereof or to a nucleic acid encoding CRF, CRF-BP, and/or CRF2R (which can be labeled). The immobilized moiety is then washed to remove any unbound material and the bound test agent or bound protein or nucleic acid is detected (e.g. by detection of a label attached to the bound molecule). The amount of immobilized label is proportional to the degree of binding between the test agent and the CRF and/or CRF-BP, and/or CRF2R and/or complexes thereof or to a nucleic acid encoding CRF, CRF-BP, and/or CRF2R.

III. Scoring the Assays.

As indicated above, methods of screening for modulators of CRF, and/or CRF-BP, and/or CRF2 receptor expression, interaction, or activity typically involve contacting a cell, tissue, organism, animal with one or more test agents and evaluating changes CRF, and/or CRF-BP, and/or CRF2 receptor nucleic acid transcription and/or translation or CRF, and/or CRF-BP, and/or CRF2 receptor protein expression or activity, or a change in CRF, and/or CRF-BP, and/or CRF2 receptor interaction(s). To screen for potential modulators, the assays described above are performed in the after administering and/or in the presence of one or more test agents using biological samples from cells and/or tissues and/or organs and/or organisms exposed to one or more test agents. The CRF, and/or CRF-BP, and/or CRF2 receptor activity and/or expression level and/or interaction is determined and, in a preferred embodiment, compared to the activity level(s) observed in “control” assays (e.g., the same assays lacking the test agent). A difference between the CRF, and/or CRF-BP, and/or CRF2 receptor expression and/or activity and/or interaction in the “test” assay as compared to the control assay indicates that the test agent is a “modulator” of CRF, and/or CRF-BP, and/or CRF2 receptor expression and/or activity and/or interaction.

In a preferred embodiment, the assays of this invention level are deemed to show a positive result, e.g. elevated expression and/or activity and/or interaction of CRF, and/or CRF-BP, and/or CRF2 receptor, when the measured protein or nucleic acid level or protein activity is greater than the level measured or known for a control sample (e.g. either a level known or measured for a normal healthy cell, tissue or organism mammal of the same species not exposed to the or putative modulator (test agent), or a “baseline/reference” level determined at a different tissue and/or a different time for the same individual). In a particularly preferred embodiment, the assay is deemed to show a positive result when the difference between sample and “control” is statistically significant (e.g. at the 85% or greater, preferably at the 90% or greater, more preferably at the 95% or greater and most preferably at the 98% or greater confidence level).

IV. High Throughput Screening

The assays of this invention are also amenable to “high-throughput” modalities. Conventionally, new chemical entities with useful properties (e.g., modulation of CRF, and/or CRF-BP, and/or CRF2 receptor expression or activity or interaction) are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A) Combinatorial Chemical Libraries

Recently, attention has focused on the use of combinatorial chemical libraries to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, 26 Dec. 1991), encoded peptides (PCT Publication WO 93/20242, 14 Oct. 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

B) High Throughput Assays of Chemical Libraries.

Any of the assays for agents that modulate expression, activity or interaction CRF, and/or CRF-BP, and/or CRF2 receptor are amenable to high throughput screening. As described above, having determined that CRF, and/or CRF-BP, and/or CRF2 receptor are associated with potentiation of NMDA receptors, it is believe that modulators can have significant therapeutic value. Certain preferred assays detect increases of transcription (i.e., increases of mRNA production) by the test compound(s), increases of protein expression by the test compound(s), or binding to the gene (e.g., gDNA, or cDNA) or gene product (e.g., mRNA or expressed protein) by the test compound(s). Alternatively, the assay can detect inhibition of the characteristic activity of the CRF, and/or CRF-BP, and/or CRF2 receptor.

High throughput assays for the presence, absence, or quantification of particular nucleic acids or protein products are well known to those of skill in the art. Similarly, binding assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

V. Kits.

In still another embodiment, this invention provides kits for practice of the assays or use of the compositions described herein. In one preferred embodiment, the kits comprise one or more containers containing antibodies and/or nucleic acid probes and/or substrates suitable for detection of CRF, and/or CRF-BP, and/or CRF2 receptor expression and/or interaction, and/or activity levels. The kits can optionally include any reagents and/or apparatus to facilitate practice of the assays described herein. Such reagents include, but are not limited to buffers, labels, labeled antibodies, labeled nucleic acids, filter sets for visualization of fluorescent labels, blotting membranes, and the like.

In another embodiment, the kits can comprise a container containing a CRF, and/or CRF-BP, and/or CRF2 receptor protein(s), and/or a vector encoding a CRF, and/or CRF-BP, and/or CRF2 receptor, and/or an sgg, and/or a cell comprising such a vector.

In addition, the kits can optionally include instructional materials containing directions (i.e., protocols) for the practice of the assay methods of this invention or the administration of the compositions described here along with counterindications. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

VI. Modulator Databases.

In certain embodiments, the agents that score positively in the assays described herein (e.g. show an ability to inhibit and/or to increase the expression and/or activity, and/or interaction of CRF, and/or CRF-BP, and/or CRF2 receptor) can be entered into a database of putative modulators CRF potentiation of NMDA receptors. The term database refers to a means for recording and retrieving information. In certain embodiments the database also provides means for sorting and/or searching the stored information. The database can comprise any convenient media including, but not limited to, paper systems, card systems, mechanical systems, electronic systems, optical systems, magnetic systems or combinations thereof. Typical databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

e like.

VII. Altering a CRF, and/or CRF-BP, and/or CRF2 Receptor Expression and/or Activity and/or Interaction.

CRF, and/or CRF-BP, and/or CRF2 receptor expression can upregulated or inhibited using a wide variety of approaches known to those of skill in the art. For example, methods of inhibiting expression include, but are not limited to antisense molecules, target-specific ribozymes, target-specific catalytic DNAs, intrabodies directed against target proteins, RNAi, gene therapy approaches that knock out CRF, and/or CRF-BP, and/or CRF2 receptor, and small organic molecules that inhibit expression of the target gene(s).

CRF, and/or CRF-BP, and/or CRF2 receptor expression and/or activity, and/or interaction can be up-regulated by introducing constructs expressing CRF, and/or CRF-BP, and/or CRF2 receptor into the cell (e.g. using gene therapy approaches) or upregulating endogenous expression of CRF, and/or CRF-BP, and/or CRF2 receptor (e.g. using agents identified in the screening assays of this invention).

In certain embodiments, CRF, and/or CRF-BP, and/or CRF2 receptor expression and/or activity and/or interaction and/or protein activity can be inhibited by the use of small organic molecules (e.g. molecules identified according to the screening methods described herein(. Such molecules include, but are not limited to molecules that specifically bind to the DNA comprising the CRF, and/or CRF-BP, and/or CRF2 receptor promoter and/or coding region, molecules that bind to and complex with CRF, and/or CRF-BP, and/or CRF2 receptor mRNA, molecules that bind to CRF, and/or CRF-BP, and/or CRF2 receptor proteins and/or complexes thereof, and the like.

The mode of administration of the CRF, and/or CRF-BP, and/or CRF2 receptor blocking or activating agent depends on the nature of the particular agent. Antisense molecules, catalytic RNAs (ribozymes), catalytic DNAs, small organic molecules, and other molecules (e.g. lipids, antibodies, etc.) used as CRF, and/or CRF-BP, and/or CRF2 receptor inhibitors can be formulated as pharmaceuticals (e.g. with suitable excipient) and delivered using standard pharmaceutical formulation and delivery methods as described below. Antisense molecules, catalytic RNAs (ribozymes), catalytic DNAs, and additionally, knockout constructs, and constructs encoding intrabodies can be delivered and (if necessary) expressed in target cells (e.g. vascular endothelial cells) using methods of gene therapy, e.g. as described below.

In order to carry out the methods of the invention, one or more inhibitors or activators of CRF, and/or CRF-BP, and/or CRF2 receptor expression and/or activity and/or interaction (e.g. ribozymes, antibodies, antisense molecules, small organic molecules, etc.) are administered to an individual to modulate NMDA receptor potentiation (e.g. to modulate a behavioral response to the consumption of alcohol and/or other substances of abuse). While this invention is described generally with reference to human subjects, veterinary applications are contemplated within the scope of this invention.

Various inhibitors may be administered, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

The active agents and various derivatives and/or formulations thereof are useful for parenteral, topical, oral, or local administration, such as by aerosol or transdermally, for prophylactic and/or therapeutic treatment of coronary disease and/or rheumatoid arthritis. The pharmaceutical compositions can be administered in a variety of unit dosage forms depending upon the method of administration. Suitable unit dosage forms, include, but are not limited to powders, tablets, pills, capsules, lozenges, suppositories, etc.

The active agent(s) and various derivatives and/or formulations thereof are typically combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers.

Other physiologically acceptable compounds include wetting agents, emulsifying agents, dispersing agents or preservatives which are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s). The excipients are preferably sterile and generally free of undesirable matter. These compositions may be sterilized by conventional, well known sterilization techniques.

The concentration of active agent(s) in the formulation can vary widely, and will be selected primarily based on fluid volumes, viscosities, body weight and the like in accordance with the particular mode of administration selected and the patient's needs.

In therapeutic applications, the compositions of this invention are administered to a patient suffering from a disease (e.g., atherosclerosis and/or associated conditions, and/or rheumatoid arthritis) in an amount sufficient to cure or at least partially arrest the disease and/or its symptoms (e.g. to reduce plaque formation, to reduce monocyte recruitment, etc.) An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms) the patient.

In certain preferred embodiments, the CRF. and/or CRF-BP, and/or CRF2 receptor modulators are administered orally (e.g. via a tablet) or as an injectable in accordance with standard methods well known to those of skill in the art. In other preferred embodiments, the CRF, and/or CRF-BP, and/or CRF2 receptor modulators can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

The foregoing formulations and administration methods are intended to be illustrative and not limiting. It will be appreciated that, using the teaching provided herein, other suitable formulations and modes of administration can be readily devised.

### EXAMPLES

The following examples are offered to illustrate, but not to limit the claimed invention.

### Example 1

### Corticotropin-Releasing Factor (CRF) Requires CRF-Binding Protein to Potentiate NMDA Receptors via CRF Receptor 2 in Dopamine Neurons

This example pertains to a study of the effects of CRF on NMDAR-mediated synaptic transmission onto dopamine neurons in an in vitro VTA slice preparation.

Methods

VTA slices from 21-29 day old C57 mice (Charles River) were prepared as previously described (Ungless et al. (2001). Nature 411: 584-587). Briefly, mice were anesthetized with halothane and then decapitated. A block of tissue containing midbrain was sliced in the horizontal plane (230 μm) in ice-cold low Ca²⁺ solution (containing in mM: 126 NaCl, 1.6 KCl, 1.2 NaH₂PO₄, 1.2 MgCl₂, 0.625 CaCl₂, 18 NaHCO₃ and 11 glucose). Slices were transferred to a holding chamber containing artificial cerebrospinal fluid (ACSF, in mM: 126 NaCl, 1.6 KCl, 1.2 NaH₂PO₄, 1.2 MgCl₂, 2.5 CaCl₂, 18 NaHCO₃ and 11 glucose) and equilibrated at 31-34° C. for at least 1 h. Picrotoxin (100 μM) was added to the ACSF for recording, to block GABAA receptor-mediated inhibitory postsynaptic potentials. All solutions were aerated with 95% O₂/5% CO₂, and perfused over the slice at a rate of 2.5 ml/min. Cells were visualized with an upright microscope using infrared differential interference contrast (IR-DIC) illumination and whole-cell voltage-clamp recordings were made with an Axopatch 1D amplifier (Axon Instruments). Electrodes (2-4 MΩ) contained in mM: 117 cesium methansulfonic acid, 20 HEPES, 0.4 EGTA, 2.8 NaCl, 5 TEA-Cl, 2.5 MgATP, 0.25 MgGTP, pH 7.2-7.4, 275-285 mOsm. Dopamine cells were identified by the presence of an Iₕ current. A bipolar stimulating electrode was placed 100-300 μm rostral to the recording electrode and was used to stimulate excitatory afferents at 0.1 Hz. Neurons were voltage-clamped at +40 mV. Coronal hippocampal slices were also prepared and maintained in a similar manner. Extracellular field potentials were recorded in the CA1 region and evoked by stimulating the stratum radiatum. All drugs were obtained from Sigma, except human/rat CRF (Tocris), Antisauvagine-30 (PolyPeptide) and CP-154,526 (a generous gift from Pfizer). CRF fragment (6-33), ovine CRF, Antisauvagine-30, CP-154,526, U-73122 and Urocortin were first dissolved in DMSO (final concentration 0.1%). EPSCs were filtered at 2 kHz, digitized at 5-10 kHz and stored using Igor Pro software (Wavemetrics). AMPAR-mediated EPSCs were recorded at −70 mV; NMDAR-mediated EPSCs were recorded at +40 mV and measured 20 msec after the stimulation artifact when the EPSC is primarily NMDAR-mediated (11,13). Statistical significance was assessed using ANOVA on 5 min bins of EPSCs, and post hoc Fisher's LSD where appropriate.

For RT-PCR (reverse transcription polymerase chain reaction) experiments, 21-29 day old C57 male mice were sacrificed as for the electrophysiology experiments, the VTA, septum and cerebellum were dissected immediately and frozen in liquid nitrogen. Total RNAs were isolated using Trizol reagent (Invitrogen) and reverse transcribed using Reverse Transcription System kit (Promega) at 42° C. for 30 minutes. CRF-R2 and control GPDH expression were analyzed by PCR with temperature cycling parameters consisting of initial denaturation at 94° C. for 2 minutes, followed by 35 cycles of denaturation at 94° C. for 30 seconds, annealing at 52° C. for 30 seconds and extension at 72° C. for 1 minute, and a final incubation at 72° C. for 7 minutes. The CRF-R2 primers were based on coding frame of mouse CRF-R2 gene: upstream, 5′-CGA GTA CTT CAA TGG CAT CAA GTA CAA-3′ (SEQ ID NO:1); and downstream, 5′-TTC GTG GTC GAT GAG TTG CAG CAG GAA-3′ (SEQ ID NO:2). The GPDH primers were based on rodent GPDH gene: upstream, 5′-TGA AGG TCG GTG TCA ACG GAT TTG GC-3′ (SEQ ID NO:3); and downstream, 5′-CAT GTA GGC CAT GAG GTC CAC CAC-3′(SEQ ID NO:4). PCR products were separated by 1.8% agarose gel and photographed by Eagle Eye II (Stratagene).

Results

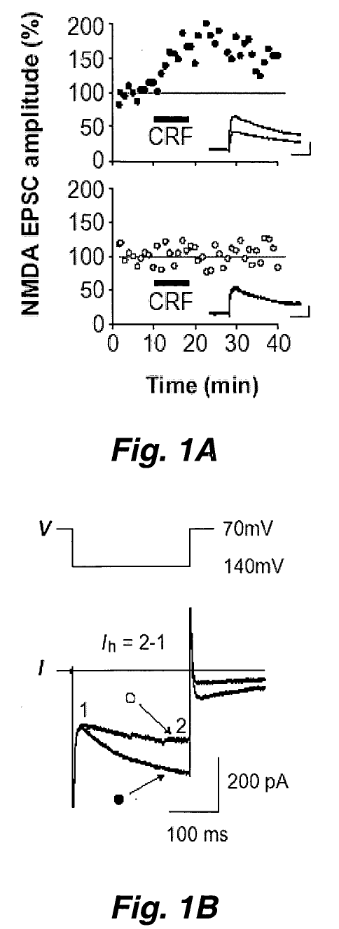

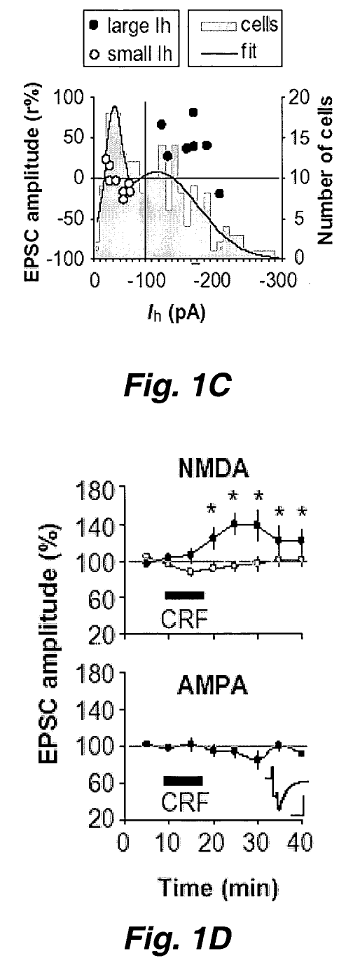

We measured excitatory postsynaptic currents (EPSCs) recorded in putative dopamine neurons in midbrain slices from mice. EPSCs were evoked while holding neurons in voltage-clamp at +40 mV; measurements were taken at a time point when the EPSC is purely NMDA-receptor-mediated (see Methods) (Bonci and Malenka (1999). J Neurosci. 19: 3723-3730; Ungless et al. (2001). Nature 411: 584-587). We then bath applied CRF at a concentration of 1 μM (8 min), which has been found to give maximal activation of cAMP in Ltk⁻ cells (Lovenberg et al. (1995). Proc. Natl. Acad. Sci. USA 92: 836-840). When CRF was applied, we observed a large, slowly developing, and transient potentiation of NMDAR-mediated EPSCs in a subset of dopamine neurons (FIG. 1A, upper panel). In some neurons, however, we did not observe any effect of CRF (FIG. 1A, lower panel), suggesting the presence of distinct sub-populations of CRF-responding and non-CRF-responding dopamine neurons. To facilitate our subsequent investigation of the effects of CRF, we therefore sought to identify a characteristic that might help us determine, a priori, to which group a particular neuron belongs. One possible electrophysiological characteristic, which has previously been shown to distinguish between distinct groups of dopamine neurons, is the size of the Iₕ (a hyperpolarization-activated inward current commonly used to identify putative dopamine neurons): calbindin-expressing dopamine neurons have a smaller Iₕ current than those that do not contain calbindin (Neuhoff et al. (2002). J. Neurosci. 10: 2385-2399). We noticed that, typically, neurons that responded to CRF had large Iₕ currents (FIG. 1B). A subsequent analysis found that Iₕ size was a strong predictor of CRF response (FIG. 1C). Moreover, a frequency histogram of 199 neurons, in which we simply measured Iₕ, shows that Iₕ distribution can be fitted well with a bimodal distribution, suggesting the existence of two subpopulations of cells that correspond to the Iₕ distribution in CRF-responding and non-CRF-responding groups (FIG. 1C, D upper panel). Henceforth, all subsequent experiments were conducted on cells with an Iₕ greater than 100 pA.

Since CRF can increase neuronal excitability at around 100 nM in the hippocampus (Aldenhoff et al. (1983). Science 221: 875-877; Blank et al. (2002). J. Neurosci. 22: 3788-3794), we tested the sensitivity of NMDAR currents to this lower concentration of CRF in the VTA. We observed a significant effect on NMDARs at this concentration, however it was smaller and slower to develop (128.01±15.7%; 25 mins after CRF onset, n=6, P<0.05). We therefore decided to conduct all subsequent experiments using a 1 μM concentration.

The observation that CRF potentiates NMDAR-mediated EPSCs can be explained by either a presynaptic effect on glutamate release, a postsynaptic modification of the NMDAR or both. If CRF acts presynaptically to increase glutamate release, then we should also observe an increase in AMPAR-mediated synaptic transmission. When we measured AMPAR-mediated EPSCs, while holding neurons in voltage-clamp at −70 mV, CRF did not potentiate these AMPAR-mediated EPSCs (FIG. 1D, lower panel). This suggests that the effect of CRF on NMDAR-mediated currents likely involves a modulation of postsynaptic NMDARs.

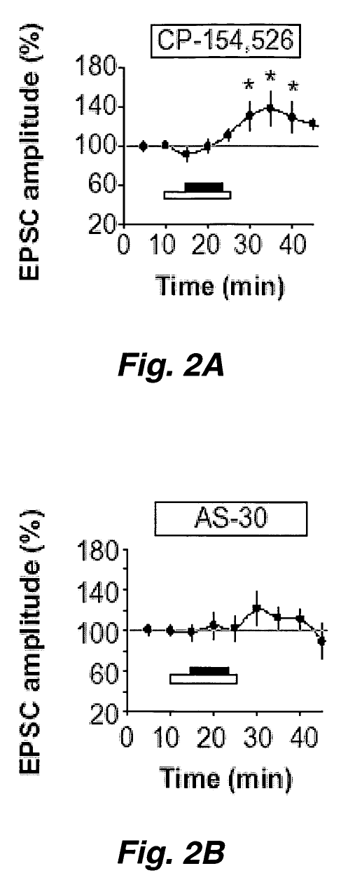

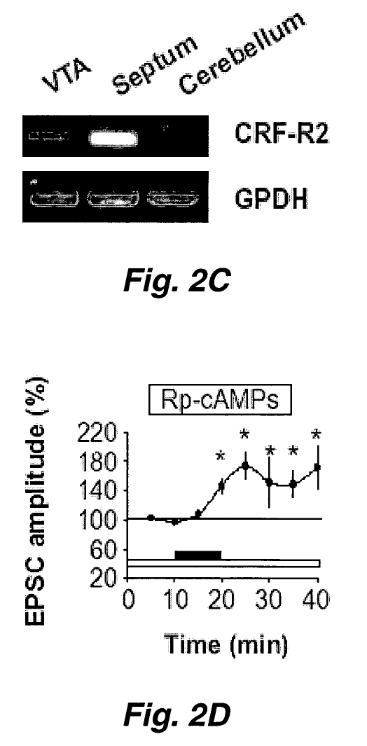

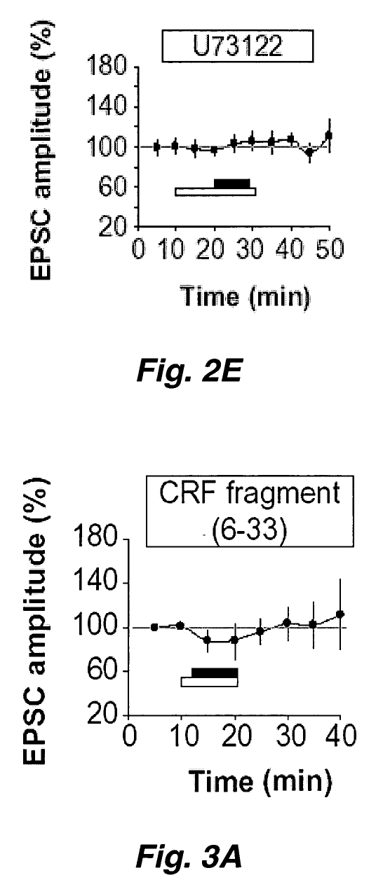

Next, we sought to determine the receptor subtype through which CRF potentiates NMDAR currents in the VTA. CRF binds to two types of receptor (5): CRF-R1 and CRF-R2. A selective CRF-R1 antagonist (CP-154,526; 3 μM for 15 min) failed to block the effects of CRF (FIG. 2a) even at this relatively high dose. However, to confirm that CP-154,526 was active, we tested its effects in the hippocampus, where CRF potentiates field potentials via CRF-R1 activation (Sillaber et al. (2002) Science 296: 931-933). In this case, the effect of CRF was blocked by co-application of 3 μM CP-154,526 (CRF 1 μM: 143.0±7.7% 15 mins after CRF onset, n=4, P<0.05; CRF plus CP-154,526: 82.9±6.9%, n=5, P>0.05). In contrast with the effect of CP-154,526 in the VTA, the presence of a selective CRF-R2 antagonist (Antisauvagine-30; 1M for 15 min), inhibited the CRF-induced potentiation in the VTA (FIG. 2B). We further tested the receptor selectivity of this effect by using a lower concentration of Antisauvagine-30 (30 nM). In the presence of this lower concentration, CRF did not significantly potentiate NMDAR EPSCs (113.9±17.7%; 15 mins after CRF onset, n=3, P>0.05). Taken together, these results strongly suggest that CRF signals through CRF-R2 to potentiate NMDAR-mediated currents in the VTA. However, although low levels of CRF-R1 mRNA have been found in the VTA using in situ hybridization techniques, CRF-R2 mRNA has been reported to be absent (Van Pett et al. (2000) J. Comp. Neurol. 428: 191-212). We therefore decided to probe the VTA for CRF-R2 mRNA using an alternative method, RT-PCR. In agreement with our pharmacological experiments, we found low levels of reliably-detectable CRF-R2 mRNA in the VTA, when compared to the septum (positive control) and cerebellum (negative control; FIG. 2C).

Next we examined how CRF-R2 activation leads to NMDAR potentiation. CRF-R2 is a seven-transmembrane-domain, G-protein-coupled receptor that can signal through a number of intracellular pathways, most notably the cAMP-PKA pathway (Dautzenberg and Hauger (2002). Trends Pharmacol. Sci. 23: 71-77). However, when we loaded the patch pipette with the cAMP inhibitor, Rp-cAMPs (100 μM), we still observed a significant potentiation of NMDAR-mediated EPSCs in the presence of CRF (FIG. 2D). In the hippocampus, the phospholipase C (PLC)-protein kinase C (PKC) pathway is involved in the effects of CRF on population spikes (Blank et al. (2002). J. Neurosci. 22: 3788-3794). Therefore, we tested whether the inhibition of this pathway interfered with the CRF-dependent potentiation of NMDA currents. When we applied the PLC inhibitor U-73122 (1 μM) for 10 min before and during CRF application, no potentiation of NMDARs was observed (FIG. 2E), suggesting that in VTA dopamine neurons, CRF potentiates NMDARs through a CRF-R2-PLC-PKC pathway.

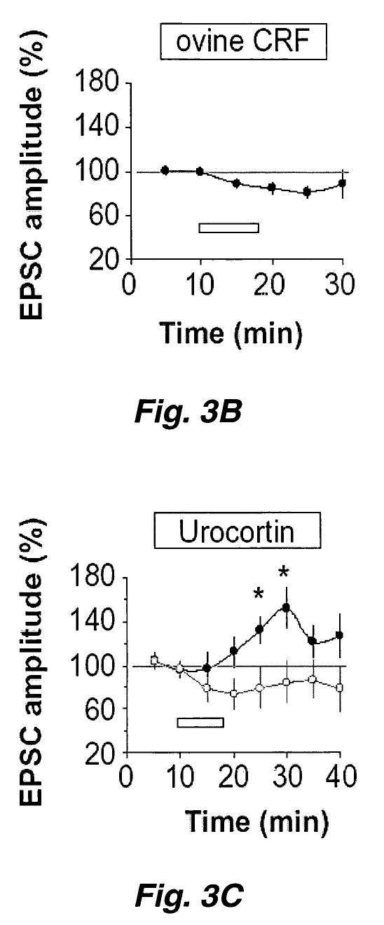

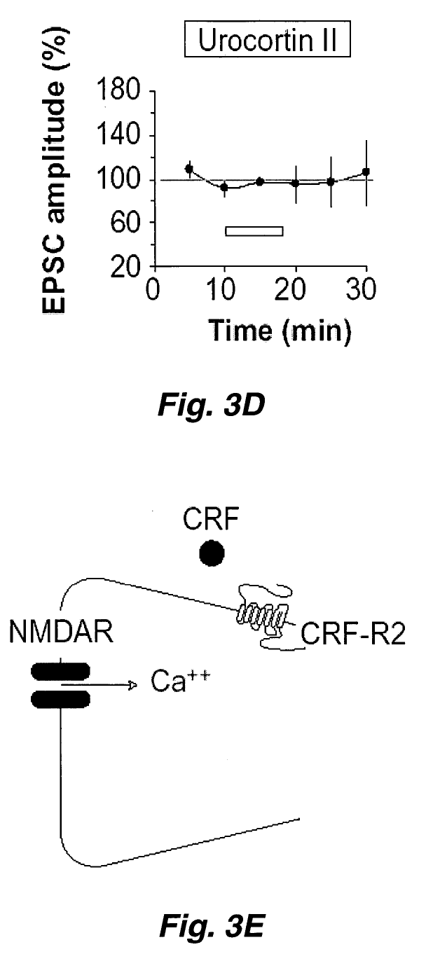

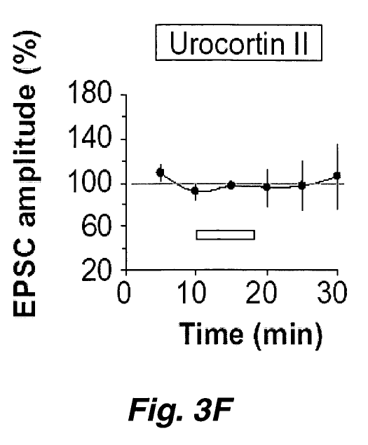

CRF also binds to a CRF-binding protein, whose role in the brain is unclear (Kemp et al. (1998). Peptides 19: 1119-1128). In the placenta, CRF-BP is thought to bind to, and essentially inactivate, CRF (Linton et al. (1990). J. Clin. Endocrinol. Metab. 70: 1574-1580). CRF-BP is also expressed in the VTA (Chan et al. (2000). Neuroscience 101: 115-129) and therefore we decided to test directly the hypothesis that CRF-BP inactivates CRF in this brain region. We co-applied CRF with a CRF fragment (6-33) (1 μM for 10 min), which shows high-affinity binding to the CRF-BP, but a much lower affinity than CRF for the CRF receptors. Consequently, the CRF fragment (6-33) competes with CRF at the CRF-BP and thereby increases the level of “free” CRF, which we expected to be reflected in an even larger increase in NMDAR-mediated EPSC size induced by CRF itself (with the possible caveat in mind that CRF may be at a saturating concentration). Contrary to this expectation, CRF did not potentiate NMDAR-mediated EPSCs in the presence of the CRF fragment (6-33) (FIG. 3A). These results suggest that CRF-induced potentiation of NMDAR-mediated EPSCs requires binding at both CRF-R2 and CRF-BP. This possibility makes the interesting prediction that ovine CRF, which differs from human/rat CRF by 7 amino acids, should not produce any potentiation of the NMDAR-mediated EPSC, because, although it binds CRF-R2 with high affinity (albeit slightly lower than that of human/rat CRF), it shows a much lower affinity for CRF-BP. Accordingly, we observed no potentiation of NMDAR-mediated EPSCs following application of ovine CRF (1 μM for 8 min; FIG. 3B). It appears, then, that CRF must be bound to CRF-BP to signal through CRF-R2 and potentiate NMDARs.

It has been suggested that the Urocortin family of CRF-like peptides may be the endogenous ligands for CRF-R2. Urocortin binds CRF-R2 and CRF-BP with higher affinity than CRF (it also binds CRF-R1 with a slightly lower affinity than CRF-R2) (Vaughan et al. (1995). Nature 378: 287-292); Urocortin II binds selectively to CRF-R2, showing low affinity for both CRF-R1 and CRF-BP (Reyes et al. (2001). Proc. Natl. Acad. Sci. USA 98: 2843-2848). In addition to this preferential binding to CRF-R2, Urocortin and Urocortin II both exhibit some anatomical overlap with CRF-R2. Given our finding that CRF must bind to CRF-R2 and CRF-BP to potentiate NMDARs, we expected Urocortin, but not Urocortin II, to mimic CRF. Accordingly, we observed potentiation of NMDAR-mediated EPSCs following application of Urocortin (1 μM for 8 min; FIG. 3C) in large Iₕ, but not small Iₕ neurons. In addition, we observed no potentiation of NMDAR-mediated EPSCs following application of Urocortin II (1 μM for 8 min; FIG. 3D). These results further confirm the necessity of CRF-BP in the ability of CRF (or CRF-like ligands) to potentiate NMDARs. When taken together, these experiments using a variety of agonists and antagonists strongly support the idea that CRF-BP plays an essential role in the synaptic actions of CRF. The results suggest that when CRF is not bound to CRF-BP it does not activate CRF-R2 (this scheme is illustrated in FIG. 3E). When CRF is bound to CRF-BP it can activate CRF-R2 and the PLC pathway to potentiate NMDARs in dopamine neurons (this scheme is illustrated in FIG. 3F).

Discussion

In this example, we demonstrate that CRF induces a potentiation of NMDAR-mediated currents in a subset of VTA dopamine neurons. Further, our experiments indicate that this potentiation is likely mediated by the R2 subtype of CRF receptors and requires the activation of a PLC-dependent pathway. In addition, we present data suggesting a novel role for the CRF-BP in the CNS. Specifically, we demonstrate that the CRF-BP is necessary for the potentiation of NMDAR-mediated currents by CRF.

The potentiation of NMDAR mediated currents is concentration dependent, with both 100 nM and 1 μM CRF producing significant increases in NMDAR currents. However, we chose to perform all subsequent studies with the 1 μM concentration since it produced a considerably more robust potentiation of NMDAR responses. Although it is possible that this concentration might be saturating, since it produces maximal activation of downstream effectors in cell cultures (Lovenberg et al. (1995). Proc. Natl. Acad. Sci. USA 92: 836-840), several lines of evidence suggest that it is unlikely that the effect of CRF observed in the present study was due to non-specific actions of CRF. For instance, the CRF-induced potentiation of NMDAR currents could be blocked by selective concentrations of a CRF-R2 antagonist. Moreover, it is likely that the concentration of CRF that actually reaches synaptic receptors in a brain slice preparation is reduced due to problems with diffusion as well as degradation of CRF by endogenous enzymes (Ritchie et al. (2003). Neuropsychopharmacology 28: 22-33). Finally, it should be noted that endogenous ligands are also released at concentrations considerably higher than receptor EC50 values derived from cells lines. For example, NMDARs exhibit EC50 values of ˜1 μM, but glutamate is estimated to be released from presynaptic terminals at a concentration of ˜1 mM (Patneau and Mayer (1990). J. Neurosci. 10: 2385-2399; Clements et al. (1992). Science 258: 1498-1501). Consequently, we think that it is very likely that applying CRF at a concentration of 1 μM mimics physiologically-relevant actions of CRF.

The current study also demonstrates that the effect of CRF on NMDAR currents in the VTA was likely mediated by the R2 subtype of CRF receptor. This is supported by the observation that a selective concentration of the R2 antagonist Antisauvagine-30 (30 nM), but not a high concentration the R1 antagonist CP 154,526 (3 μM), inhibited the response to CRF in the VTA. This result is surprising since previous studies, using in situ hybridization, have demonstrated the presence of the R1 but not the R2 subtype of receptor in the VTA (Van Pett et al. (2000) J. Comp. Neurol. 428: 191-212). However, the pharmacological data in the current study were supported by results obtained by RT-PCR that demonstrated a low level of expression of CRF-R2 mRNA in the VTA. The low level of expression of mRNA for the R2 subtype of receptor in the VTA may also be consistent with the observation that CRF potentiated NMDAR currents only in a subset of cells within this brain region.

Furthermore, the observation that the CRF-BP is required for CRF to potentiate NMDAR currents in the VTA is quite striking, since it appears to act merely as a CRF buffer in the placenta (Linton et al. (1990). J. Clin. Endocrinol. Metab. 70: 1574-1580). However, in the brain, there are several reasons to suppose that CRF-BP may have additional roles. In particular, intracerebroventricular injections of CRF-BP inhibitors have behavioral and neural effects, some of which are inconsistent with them simply elevating CRF levels (Chan et al. (2000). Neuroscience 101: 115-129; Heinrichs and Joppa (2001). Behav. Brain Res. 122: 43-50). For example, although CRF-BP inhibitors produce arousal effects similar to CRF itself, they do not produce the anxiety phenotype associated with high levels of CRF (Heinrichs and Joppa (2001). Behav. Brain Res. 122: 43-50).

Further investigations will elucidate the mechanism through which the binding between CRF and the CRF-BP facilitates the actions of CRF at CRF-R2. One possibility is that CRF-BP plays a permissive role, and facilitates the binding of CRF to the CRF-R2. An alternative possibility is that the CRF-BP increases the time that CRF spends in proximity to the CRF-R2. A further possibility concerns the observation that when CRF binds to CRF-BP it forms a stable dimer, and under some circumstances it appears to undergo conformational modifications (Lowry et al. (1996). J. Mol. Endocrinol. 16: 39-44). It might be this dimerization and/or conformational change that allows CRF to signal specifically through the PLC pathway when bound to CRF-R2.

Under physiological circumstances, activation of NMDARs in the VTA plays a key role in the switch from regular firing to burst firing in dopamine neurons (Overton and Clark (1997). Brain Res. Brain Res. Rev. 25: 312-334). Burst firing leads to substantial increases in dopamine levels at projection areas including the nucleus accumbens, prefrontal cortex and amygdala. Neuropeptides such as neurotensin are also preferentially co-released under these conditions (Bean, A. J. & Roth, R. H. (1991). J Neurosci. 11, 2694-2702). The potentiation of NMDARs in dopamine neurons by CRF would therefore be expected to increase burst firing, and may be involved in stress-induced activation of the dopamine system, which could in turn modulate drug-taking behavior (Sarnyai et al. (2001). Pharmacol. Rev. 53: 209-243).

NMDARs in the VTA also play a crucial role in the induction of long-term synaptic plasticity at excitatory synapses (Bonci and Malenka (1999). J Neurosci. 19: 3723-3730; Overton et al. (1999). Neuroreport 10: 221-226; Ungless et al. (2001). Nature 411: 584-587). Recent in vitro studies have shown that these excitatory synapses can be bi-directionally modulated, and develop long-term changes in synaptic transmission such as NMDA-receptor-dependent LTP (Bonci and Malenka (1999). J Neurosci. 19: 3723-3730; Overton et al. (1999). Neuroreport 10: 221-226; Ungless et al. (2001). Nature 411: 584-587; Overton and Clark (1997). Brain Res. Brain Res. Rev. 25: 312-334). Furthermore, a single in vivo cocaine exposure can induce LTP-like plasticity in dopamine neurons, the induction of which can be blocked by NMDAR antagonists (Ungless et al. (2001). Nature 411: 584-587). This, as well as previous studies, suggest that LTP in the VTA may represent a common site of action for the long-term consequences of both stress and drugs of abuse (Clark and Overton (1998). Addict. Biol. 3: 109) and play a role in the modulation of drug taking behavior by stress. Consistent with this possibility, repeated stress or cocaine increases the expression of NMDAR and AMPAR subunits in the VTA, and stress also produces plasticity at excitatory synapses onto these neurons (Fitzgerald et al. (1996). J Neurosci. 16: 274-282; Saal et al. (2003) Neuron 37: 577-582). Although the role of CRF in such stress-dependent plasticity at AMPARs has not been investigated yet, our data indicates that the potentiation of NMDARs by CRF in the VTA during stress may facilitate the induction of LTP-like processes.

As mentioned previously, the subset of neurons that do not respond to CRF may represent calbindin-expressing (or low Iₕ) dopamine neurons (Neuhoff et al. (2002). J Neurosci. 10: 2385-2399). These neurons have also been shown to co-express neuropeptides, such as neurotensin (German, D. C. & Liang, C-L. (1993). Neuroreport 4, 491-494). Consequently, CRF might be expected to increase dopamine release, without a concurrent increase in neurotensin release. Since neurotensin is thought to act as an endogenous antipsychotic (Nemeroff (1980). Biological Psychiatry 15: 283-302), changes in the dopamine/neurotensin ratio may be important for the development and subsequent treatment of psychosis (Gariano and Groves (1989). Biological Psychiatry 26: 303-14). It is of note, therefore, that CRF levels are elevated in schizophrenic patients following withdrawal from antipsychotic medication (Forman, (1994). Schizophr. Res. 12: 43-51).

Whatever the broader implications may be for the effects of CRF on the dopamine system, the present findings open new avenues for potential therapeutic targets in disorders where CRF and/or dopamine levels are decreased, since such a ligand might lack the anxiogenic properties of other CRF-like peptides that bind the R1 subtype of CRF receptor.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of screening for an agent that modulates corticotrophin-releasing factor (CRF) potentiation of N-methyl-D-aspartate receptor (NMDAR) mediated currents, said method comprising:
contacting a cell with a test agent; and
detecting the activity or expression of a CRF2 receptor;
wherein an alteration of expression or activity of a CRF2 receptor as compared to a control indicates that said test agent is an agent that modulates CRF potentiation of NMDAR-mediated currents.

2. The method of claim 1, wherein said cell is a nerve cell.

3. The method of claim 1, wherein said cell is a cell in a neurological tissue.

4. The method of claim 1, wherein said cell is a cell in a brain slice preparation

5. The method of claim 1, wherein said cell is a nerve cell in culture.

6. The method of claim 1, wherein said detecting comprises detecting an electrophysiological signal from a neurological cell.

7. The method of claim 1, wherein said detecting comprises detecting an electrophysiological signal from a dopamine neuron.

8. The method of claim 1, wherein said detecting comprises detecting an electrophysiological signal from a dopamine neuron in a ventral tegmental area (VTA).

9. The method of claim 1, wherein said detecting comprises detecting a CRF2 receptor nucleic acid.

10. The method of claim 9, wherein said detecting comprises a nucleic acid hybridization.

11. The method of claim 10, wherein said detecting comprises a method selected from the group consisting of a Northern blot, a Southern blot using DNA derived from a CRF2 receptor RNA, an array hybridization, an affinity chromatography, and an in situ hybridization.

12. The method of claim 1, wherein said detecting comprises detecting a CRF2 receptor protein.

13. The method of claim 12, wherein said detecting comprises binding a CRF receptor protein with a detectable label.

14. The method of claim 12, wherein said detecting is via a method selected from the group consisting of capillary electrophoresis, a Western blot, mass spectroscopy, ELISA, immunochromatography, and immunohistochemistry.

15. The method of claim 1, wherein said control comprises a cell contacted with said test agent at a lower concentration.

16. The method of claim 1, wherein said control comprises a cell not contacted with said test agent.

17. A method of screening for an agent that modulates corticotrophin-releasing factor (CRF) potentiation of N-methyl-D-aspartate receptor (NMDAR) mediated currents, said method comprising:
contacting a cell with a test agent; and
detecting the activity or expression of a CRF Binding Protein 1 (CRFBP1);
wherein an alteration of expression or activity of a CRFBP1 as compared to a control indicates that said test agent is an agent that modulates CRF potentiation of NMDAR-mediated currents.

18. The method of claim 17, wherein said screening further comprises contacting said cell with exogenous CRF.

19. The method of claim 17, wherein said cell is a nerve cell.

20. The method of claim 17, wherein said cell is a cell in a neurological tissue.

21. The method of claim 17, wherein said cell is a cell in a brain slice preparation

22. The method of claim 17, wherein said cell is a nerve cell in culture.

23. The method of claim 17, wherein said detecting comprises detecting an electrophysiological signal from a neurological cell.

24. The method of claim 17, wherein said detecting comprises detecting an electrophysiological signal from a dopamine neuron.

25. The method of claim 17, wherein said detecting comprises detecting an electrophysiological signal from a dopamine neuron in a ventral tegmental area (VTA).

26. The method of claim 17, wherein said detecting comprises detecting a CRF2 receptor nucleic acid.

27. The method of claim 26, wherein said detecting comprises a nucleic acid hybridization.

28. The method of claim 27, wherein said detecting comprises a method selected from the group consisting of a Northern blot, a Southern blot using DNA derived from a CRF2 receptor RNA, an array hybridization, an affinity chromatography, and an in situ hybridization.

29. The method of claim 17, wherein said detecting comprises detecting a CRF2 receptor protein.

30. The method of claim 29, wherein said detecting comprises binding a CRF receptor protein with a detectable label.

31. The method of claim 29, wherein said detecting is via a method selected from the group consisting of capillary electrophoresis, a Western blot, mass spectroscopy, ELISA, immunochromatography, and immunohistochemistry.

32. The method of claim 17, wherein said control comprises a cell contacted with said test agent at a lower concentration.

33. The method of claim 17, wherein said control comprises a cell not contacted with said test agent.

34. A method of screening for an agent that modulates the activity of corticotrophin-releasing factor (CRF) on a dopaminergic neuron, said method comprising:
contacting a test agent to one or more components of CRF signaling selected from the group consisting of a CRF, a CRF-BP, and a CRF2 receptor;
and detecting an increase or decrease in interaction between said CRF and said CRF BP and/or said CRF and said CRF2 receptor where an increase or decrease in said interaction, as compared to a control, indicates that said test agent modulates the activity of corticotrophin-releasing factor (CRF) on a dopaminergic neuron.

35. The method of claim 34, wherein said detecting comprises detecting via a two-hybrid system.

36. The method of claim 34, wherein said detecting comprises detecting via a gel-shift assay.

37. The method of claim 34, wherein said detecting comprises detecting specific binding of said test agent to one or more of said components.

38. The method of claim 34, wherein said interaction is in vivo.

39. The method of claim 34, wherein said interaction is ex vivo.

40. The method of claim 34, wherein said interaction is in a cultured neural cell.

41. The method of claim 34, wherein said interaction is in a brain slice preparation.

42. The method of claim 34, wherein said test agent is not an antibody.

43. The method of claim 34, wherein said test agent is not a protein.

44. The method of claim 34, wherein said test agent is a small organic molecule.

45. A method of modulating the activity of corticotrophin-releasing factor (CRF) on a dopaminergic neuron in a mammal, said method comprising:
modulating binding between crf and crfBP and/or
modulating binding between crf and crf2 receptor.

46. The method of claim 45, wherein said modulating comprises:
inhibiting binding between crf and crfBP; and/or
inhibiting binding between crf and crf2 receptor.

47. The method of claim 45, wherein said modulating comprises:
inhibiting expression or activity of one or more components selected from the group consisting of crf, crfBP1, and a CRF2 receptor.

48. The method of claim 45, wherein said modulating comprises:
enhancing binding between crf and crfBP and/or
enhancing binding between crf and crf2 receptor.

49. The method of claim 45, wherein said modulating comprises:
enhancing expression or activity of one or more components selected from the group consisting of crf, crfBP1, and a CRF2 receptor.

50. The method of claim 45, wherein said modulating comprises reducing the level of stress experienced by said mammal.

51. A method of mitigating one or more symptoms associated with chronic consumption of a substance of abuse or with withdrawal from such chronic consumption, said method comprising:
inhibiting interaction between CRF and CRFBP1 and/or a CRF2 receptor.

52. A method of enhancing long term potentiation, said method comprising:
enhancing interaction between CRF and CRFBP1 and/or a CRF2 receptor.

53. A method of mitigating a symptom associated with Alzheimer's disease or Parkinson's disease, said method comprising:
enhancing interaction between CRF and CRFBP1 and/or a CRF2 receptor.

54. A method of decoupling CRF from glutamate receptor activity, said method comprising:
inhibiting interaction between CRF and CRFBP1 and/or a CRF2 receptor.

55. A method of modulating the activity of crf on a dopaminergic neuron, said method comprising:
modulating binding between crf and crfBP; and/or
modulating binding between crf and crf2 receptor.

56. A method of screening for a molecule that enhances cognition, said method comprising
contacting a cell, tissue or organism with a test agent; and
detecting activity of the test agent at the CRF1 receptor and the CRF2 receptor, wherein an agent that inhibits CRF1 receptor and agonizes the CRF2 receptor is a cognitive enhancer.
